(19) United States
(12) Patent Application Publication
(10) Pub. No.: US 2009/0042179 A1
(43) Pub. Date: Feb. 12, 2009

(54) Fluorescence Reflection Imaging Device with Two Wavelengths

(75) Inventors: Philippe Peltie, Saint Paul De Varces (FR); Michel Berger, Claix (FR); Roland Sauze, Grenoble (FR); Stephane Bonnet, Seyssinet (FR)

Correspondence Address: OLIFF & BERRIDGE, PLC, P.O. BOX 320850, ALEXANDRIA, VA, 22320-4850, US

(73) Assignee: Commissariat A L'Energie Atomique, Paris (FR)

(21) Appl. No.: 11/795,975

(22) PCT Filed: Jan. 20, 2006
(86) PCT No.: PCT/FR2006/000131

§ 371 Date: Jul. 25, 2007

(30) Foreign Application Priority Data
Feb. 14, 2005 (FR) 0501471

(51) Int. Cl. C12Q 1/00; G01J 3/42; G06T 7/40

(52) U.S. Cl. 435/4; 356/320; 382/120

(57) ABSTRACT

A first light source has a first wavelength corresponding to an excitation wavelength of a fluorophore. The excitation wavelength and an emission wavelength of the fluorophore delineate a predetermined interval. A second light source has a second wavelength offset with respect to the first wavelength so as to be outside said predetermined interval. The offset between the first and second wavelengths is comprised between 30 nm and 100 nm. A camera comprises a filter opaque to the first and second wavelengths and transparent to the emission wavelength and to wavelengths substantially higher than the higher of the first and second wavelengths. The light sources and camera are synchronized to alternately activate one of the light sources and make the camera alternately acquire a fluorescence image and a background noise image.

### BACKGROUND OF THE INVENTION

The invention relates to a fluorescence reflection imaging device comprising at least a first light source of a first wavelength corresponding substantially to an excitation wavelength of a marking fluorophore presenting a main emission wavelength, the excitation wavelength and the main emission wavelength delineating a predetermined interval, the device comprising a camera and at least a second light source of a second wavelength, offset with respect to the first wavelength, so that the second wavelength is outside said predetermined interval.

### STATE OF THE ART

Fluorescence Reflection Imaging (FRI) is a widely used technique for in vivo fluorescence. It consists in performing fluorescence imaging of zones marked by a fluorophore more often than not coupled with an antibody which fixes itself specifically on unhealthy tissues or organs, for example cancerous tissues. The FRI technique is also used for in vitro imaging, for example for reading biochips. The fields involved are both vegetal and animal biology. For example, the FRI technique can be implemented to monitor the progression of viruses marked by a fluorophore in plants. In addition, techniques using several markers have been proposed.

For in vitro imaging, for example reading biochips, the traditional readers are epi-illumination microscopes equipped with CCD cameras and generally adapted to fields of vision of small size of about a few square millimeters.

For in vivo imaging, two approaches are generally encountered. A first approach, typically used for small animal imaging (mice, rats, etc.), consists in using a CCD camera equipped with an auxiliary lighting device, for example an annular or incidence lighting device. The corresponding equipment is generally bulky. A second approach, designed for use in the human body, consists in using an endoscope at the end of which a camera and a lighting system are connected. The endoscopes are generally limited to wavelengths in the visible range, near-ultraviolet and near-infrared being their transmission limit.

Numerous devices exist enabling an operation area to be visualized, in particular devices using different spectral bands to obtain two images which are subsequently superposed. For example, an excitation light of a fluorophore which marks biological tissues and a white light to have a realistic vision of the operation area. The methods used generally present problems of shaping of the lighting beam and of autofluorescence.

The document US2001/007920, for example, describes an endoscopy system comprising an illumination unit comprising a white light source and an excitation light laser source used to excite a fluorescence image of a living body. The system further comprises a fluorescence image detection unit comprising a CCD detector and a conventional image detection unit comprising a CCD detector. The two images are superposed by means of superposition means. The two light sources are activated alternately, each causing exposure of the associated detector by the corresponding light.

The document U.S. Pat. No. 6,537,211, for example, describes an imaging system of a surface of cancerous tissues comprising a white light source to excite a reflection image and a UV light source to excite a fluorescence image. The two images are detected alternately by a common CCD camera and displayed on a common screen. The fluorescence excitation light is blocked when the white light is used for illumination and vice versa.

When a target marked by a fluorophore is detected, one is generally troubled by the autofluorescence of the tissues and by the light diffusion due to the tissues, in particular when the targets are located at a depth of more than 1 mm. These problems are all the greater the shorter the wavelengths of the measurements, for example in the blue, and less troublesome in the infrared. However, in the infrared, the efficiency of the detectors is low and the choice of fluorophores is limited. Work is therefore essentially performed in an excitation band comprised between 480 nm and 780 nm with an emission band comprised between 520 nm and 800 nm. Moreover, the diffusion and autofluorescence interference signal is not at all stationary.

The document U.S. Pat. No. 5,741,648 describes a method for analyzing fluorescence images of cells provided with fluorescent markers. The document describes a technique for determining the autofluorescence of a sample comprising the marked cells and illuminated with a first wavelength. A second excitation wavelength is chosen in the tail end of the fluorescent marker excitation spectrum. Irradiation with the second wavelength essentially causes autofluorescence of the sample, whereas the first wavelength causes a strong excitation of the fluorescent markers. Grey levels corresponding to autofluorescence are subtracted from the grey levels corresponding to excitation with the first wavelength in order to determine the fluorescence due to the fluorescent markers. However, the result obtained presents a strong background noise.

### OBJECT OF THE INVENTION

One object of the invention is to remedy these shortcomings and in particular to enable rapid and precise location of marked cells in biological tissues and in particular to reduce the measurement noise.

According to the invention, this object is achieved by the appended claims and more particularly by the fact that the offset between the first and second wavelengths being comprised between 30 nm and 100 nm, the camera comprises means for filtering at least the first and second wavelengths, the means for filtering being transparent to the main emission wavelength and to wavelengths substantially higher than the higher of the first and second wavelengths, the device comprising means for synchronizing the first and second light sources and the camera to alternately activate one of the first and second light sources and make the camera alternately acquire a fluorescence image and a background noise image.

It is a further object of the invention to provide a method for visualizing an object using an imaging device according to the invention and comprising a marking step of the cells by the marking fluorophore, and alternately a first step of activation of the first light source and acquisition of a fluorescence image, and a second step of activation of the second light source and acquisition of a background noise image.

### BRIEF DESCRIPTION OF THE DRAWINGS

Other advantages and features will become more clearly apparent from the following description of particular embodiments of the invention given for non-restrictive example purposes only and represented in the accompanying drawings, in which:

FIGS. 1 and 2 respectively represent a part in side view and another part in top view of a particular embodiment of a device according to the invention.

FIGS. 3 and 4 respectively represent the transmission curves of two particular embodiments of a filter versus the wavelength λ.

FIG. 5 illustrates synchronization of the activation times of the first light source, of the second light source and of the camera versus time.

### DESCRIPTION OF PARTICULAR EMBODIMENTS

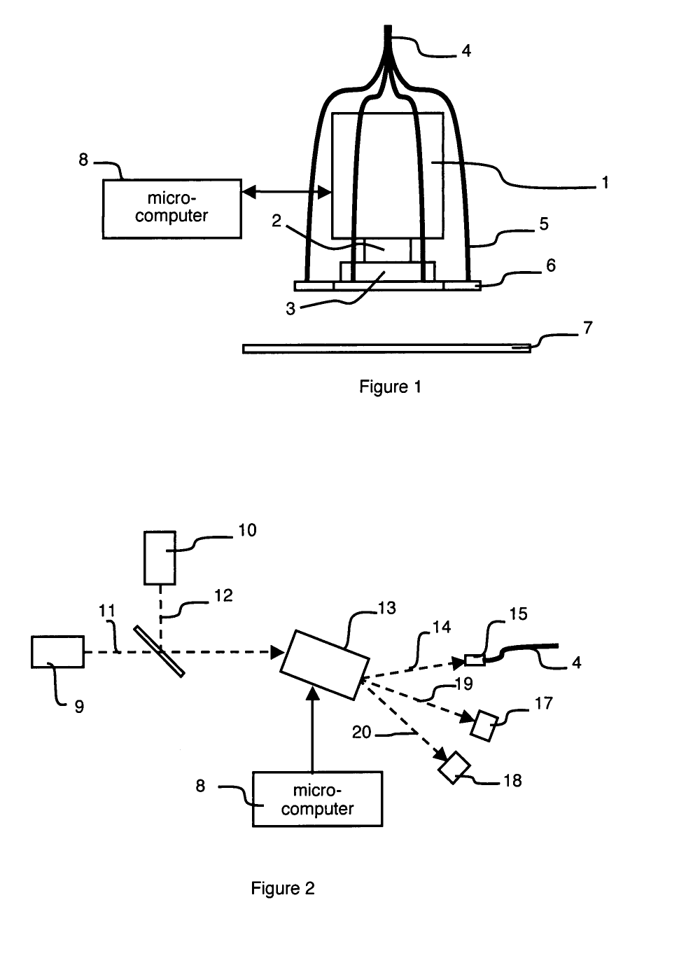

In FIG. 1, the imaging device comprises a camera 1 equipped with a lens 2 and a filter 3. In the particular embodiment represented in FIG. 2, light is conveyed to the camera 1 by a main optic fiber 4 which is connected to a plurality of optic fibers 5 attached to a ring-shaped light diffuser 6 enabling an object arranged on an object support 7, for example a biological sample, to be uniformly illuminated. The object can also be an operation area. The object is marked by a marking fluorophore. The camera detects the light emitted by the object by means of the filter 3 and transmits signals representative of images to a processing unit, for example a microcomputer 8.

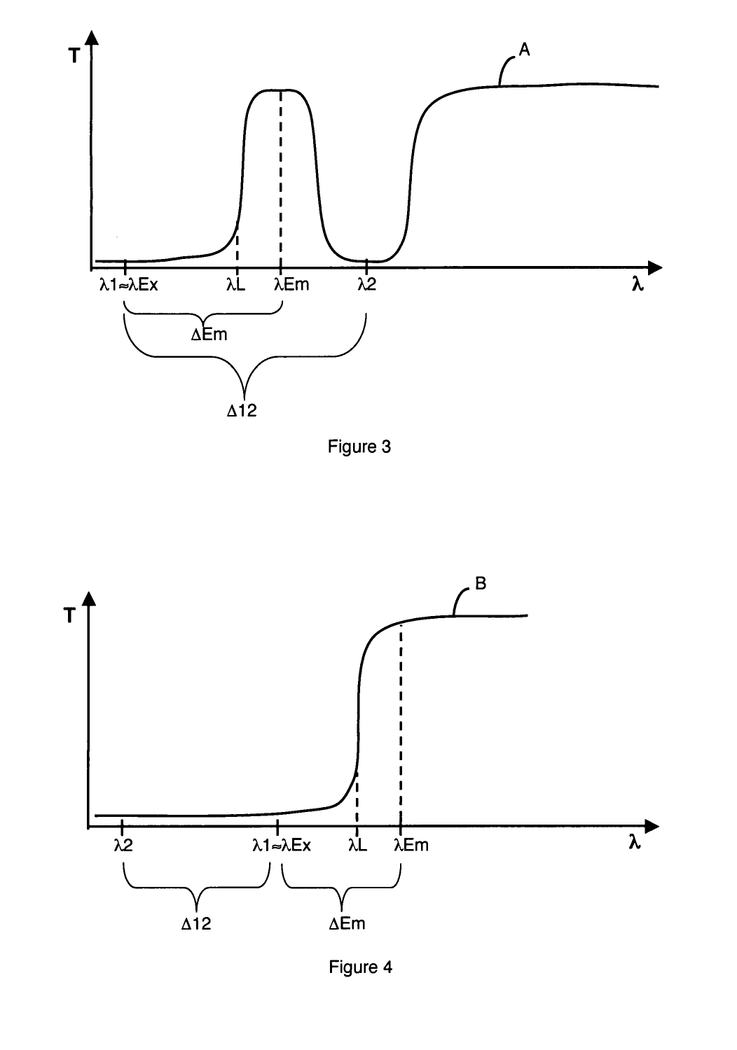

A first light source 9 and a second light source 10 are represented in FIG. 2. The first light source 9 emits a first light beam 11 having a first wavelength λ1 corresponding substantially to an excitation wavelength λEx of the marking fluorophore. The marking fluorophore presents a main emission wavelength λEm. The excitation wavelength % Ex and the main fluorescence emission wavelength λEm delineate a predetermined interval ΔEm. The second light source 10 produces a second light beam 12 of a second wavelength λ2 offset with respect to the first wavelength λ1. The offset Δ12 (FIGS. 3 and 4) between the second wavelength λ2 and the first wavelength λ1 is comprised between 30 nm and 100 nm. The second wavelength λ2 is then relatively close to the first wavelength λ1. Thus, the light from the second light source 10 causes autofluorescence of the illuminated object almost under the same conditions as the light from the first light source 9, without causing fluorescence of the marking fluorophore which has an offset ΔEm of less than 30 nm. The two wavelengths therefore cause almost the same autofluorescence signal, which constitutes a background noise of the fluorescence signal of the marking fluorophore.

The first and second light beams 11 and 12 are preferably directed to the input of an acousto-optic tunable filter 13 (AOTF), enabling the wavelength of a selection beam 14 directed to an input 15 of the optic fiber 4 to be selected. The selection beam 14 therefore has a well-determined wavelength, in particular either the first wavelength λ1 or the second wavelength λ2. The acousto-optic tunable filter 13 is controlled by a control unit, for example formed by the microcomputer 8, which also performs image processing.

In FIG. 2, first and second photodiodes 17 and 18 enable the intensity of reference beams 19 and 20 corresponding respectively to the first and second wavelengths λ1 and λ2 to be detected. When the wavelength of the selection beam 14 is the first wavelength λ1, a first reference beam 19 corresponding to the zero order of the AOTF 13 is in fact incident on the first photodiode 17 and enables the intensity emitted by the first light source 9 to be determined. A second reference beam 20 corresponding to the zero order of the second wavelength λ2 is detected by the second photodiode 18 and thus enables the intensity emitted by the second light source 10 to be determined.

In the particular embodiment corresponding to FIG. 3, the second wavelength λ2 is higher than the first wavelength λ1. The second wavelength λ2 must be outside the predetermined interval ΔEm delineated by the excitation wavelength λEx and the main emission wavelength λEm of the marking fluorophore. As illustrated by the transmission curve A of the filter 3 represented in FIG. 3, the filter 3 is opaque to the first (λ1) and second (λ2) wavelengths, which enables back-scattering of the excitation light to be filtered. The filter 3 is transparent to the main emission wavelength λEm. The filter 3 must be transparent to wavelengths substantially higher than the higher of the first and second wavelengths λ1 and λ2, i.e. than the second wavelength λ2, in the embodiment corresponding to FIG. 3. The wavelengths higher than the higher of the first and second wavelengths λ1 and λ2 correspond in particular to autofluorescence interference signals of the object.

For example, the first and second wavelengths λ1 and λ2 can be respectively 488 nm and 532 nm (offset Δ12=44 nm) when the fluorophore used is fluorescein, 540 nm and 600 nm (offset Δ12=60 nm) when the fluorophore used is Cy3, and 633 nm and 690 nm (offset Δ12=57 nm) when the fluorophore used is Cy5.

The filter 3 corresponding to FIG. 3 preferably comprises a high-pass filter having a limit wavelength λL (FIG. 3) located between the first wavelength λ1 and the main emission wavelength λEm. When the fluorophore used is fluorescein, the limit wavelength λL is for example 500 nm. Moreover, in the embodiment illustrated in FIG. 3, the filter 3 comprises a band-stop filter blocking a narrow spectral band (for example a notch type holographic filter), corresponding in particular to the second wavelength λ2. The band-stop filter is superposed on the high-pass filter.

As the light sources always present a certain emission width, the difference between the first wavelength λ1 and the limit wavelength λL must be substantially greater than the emission width of the first light source 9 to ensure that the corresponding diffusion light is filtered by the filter 3. For the same reason, the spectral band blocked by the band-stop filter must be higher than the emission width of the second light source 10.

In the particular embodiment corresponding to FIG. 4, the second wavelength λ2 is lower than the first wavelength λ1. As in the previously described embodiment, the second wavelength λ2 must be outside the predetermined interval ΔEm, delineated by the excitation wavelength λEx and the main emission wavelength λEm of the marking fluorophore. As illustrated by the transmission curve B of the filter 3 represented in FIG. 4, the filter 3 is, as before, opaque to the first (λ1) and second (λ2) wavelengths and transparent to the main emission wavelength λEm. The filter 3 is transparent to wavelengths substantially higher than the higher of the first and second wavelengths λ1 and λ2, i.e. to the first wavelength λ1 in the embodiment corresponding to FIG. 4.

The filter 3 corresponding to FIG. 4 preferably comprises a high-pass filter having a limit wavelength λL located between the first wavelength λ1 and the main emission wavelength λEm.

The difference between the first wavelength λ1 and the limit wavelength λL must, as before, be substantially greater than the emission width of the first light source 9 to ensure that the corresponding diffusion light is filtered by the filter 3.

According to the invention, the first (9) and second (10) light sources and the camera 1 are synchronized in such a way as to alternately activate one of the first (9) and second (10) light sources and to make the camera 1 alternately acquire a fluorescence image and a background noise image. The fluorescence image comprises the fluorescence signal emitted by the marking fluorophore on the one hand, and interference signals due to the autofluorescence on the other hand, whereas the background noise image essentially comprises signals due to the autofluorescence of the tissues and to the interference fluorescences (filters, various opticals, object support when in vitro reading is involved).

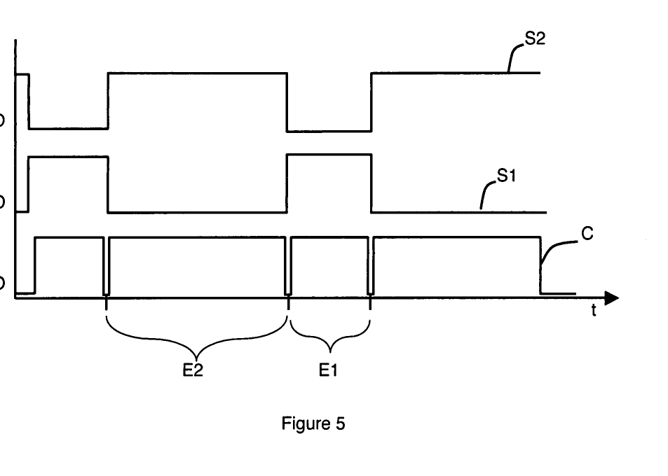

Synchronization can be performed by means of the microcomputer 8 (FIGS. 1 and 2) and of the acousto-optical tunable filter 13. Thus, as represented in FIG. 5, the first light source 9 is activated (the curve S1 then takes the value I in FIG. 5) while the second light source 10 is deactivated (the curve S2 then takes the value O in FIG. 5) and vice versa, whereas the camera 1 is activated (the curve C takes the value I) respectively for acquisition of images from each of the sources. Between two image acquisitions, the camera is deactivated (the curve C takes the value O) during a very short time of about one microsecond.

The integration time of each type of image, fluorescence image and background noise image, are not necessarily the same. The camera can be controlled with a variable integration time, either in programmed manner or manually, for example by the doctor. For example, in FIG. 5, the illumination time of the second light source 10 (S2) is longer than the illumination time of the first light source 9 (S1) and the integration time of the camera associated with the background noise image is therefore longer than the integration time associated with the fluorescence image.

The background noise image and the fluorescence image can for example be displayed at the same time on the same monitor. Any combination of the background noise image and the fluorescence image can also be determined and displayed, for example via the computer 8. In a particular embodiment, the background noise image is subtracted from the corresponding fluorescence image. In another particular embodiment, the fluorescence image is divided by the corresponding background noise image. Furthermore, the background noise image enables the whole of the illuminated object, for example an operation area, to be displayed.

A method for displaying an object using the device according to the invention comprises a step of marking cells by means of the marking fluorophore. The method then alternately comprises a first step E1 of activation of the first light source 9 and acquisition of a fluorescence image, and a second step E2 of activation of the second light source 10 and acquisition of a background noise image, as illustrated in FIG. 5.

Even if the second wavelength λ2 is relatively close to the first wavelength λ1, the autofluorescence signal of the areas not marked by the marking fluorophore does not necessarily have the same intensity. In a particular embodiment of the method according to the invention, durations of the first E1 and second E2 steps are adjusted so that an image area corresponding to a predetermined area not marked by the marking fluorophore presents the same luminosity on a fluorescence image and on a background noise image.

Thus, when a background noise image is subtracted from a fluorescence image, an image essentially representing the signal due to the marking fluorophore is obtained. When the fluorescence image is divided by the background noise image, an accentuated representation of the areas marked by the fluorophore is obtained.

It is therefore in particular the autofluorescence signal at higher wavelengths than the higher of the first and second wavelengths λ1 and λ2 that makes it possible to characterize the autofluorescence signal that is detected at the emission wavelength λEm and that can not be filtered without reducing the fluorescence signal at the emission wavelength λEm.

Adjustment of the durations of the first E1 and second E2 steps can for example be performed by acquisition of a fluorescence image and of a background noise image with the same acquisition time and calculation of luminosity histograms on a predetermined portion of image, corresponding to a non-marked area. The maximums of the histograms then enable a ratio of the durations of the first E1 and second E2 image acquisition steps to be determined automatically, enabling the same luminosity to be obtained on a fluorescence image and on a background noise image.

The intensity of the selection beam 14 can also be adjusted for each of the first and second wavelengths by means of the acousto-optic tunable filter so that an image zone corresponding to a predetermined area not marked by the marking fluorophore presents the same luminosity on a fluorescence image and on a background noise image.

The invention is not limited to the embodiments represented. In particular, several first and several second light sources can be used, for example four sources respectively having wavelengths of 488 nm and 532 nm on the one hand, and 633 nm and 690 nm on the other hand, the fluorophores used being fluorescein and Cy5. Thus, the filter 3 is opaque to these four wavelengths and transparent to the two fluorophore emission wavelengths and to wavelengths substantially higher than the higher of the first and second wavelengths, i.e. above 690 nm.

1-12. (canceled)

13. A fluorescence reflection imaging device comprising at least a first light source of a first wavelength corresponding substantially to an excitation wavelength of a marking fluorophore presenting a main emission wavelength, the excitation wavelength and the main emission wavelength delineating a predetermined interval, the device comprising a camera and at least a second light source of a second wavelength, offset with respect to the first wavelength so that the second wavelength is outside said predetermined interval, wherein, the offset between the first and second wavelengths being comprised between 30 nm and 100 nm, the camera comprises filtering means for filtering at least the first and second wavelengths, the filtering means being transparent at the main emission wavelength and at wavelengths substantially higher than the higher of the first and second wavelengths, the device comprising synchronization means for synchronizing the first and second light sources and the camera to alternately activate one of the first and second light sources and make the camera alternately acquire a fluorescence image and a background noise image.

14. The device according to claim 13, comprising means for subtracting the fluorescence image and the background noise image.

15. The device according to claim 13, comprising means for dividing the fluorescence image by the background noise image.

16. The device according to claim 13, wherein the second wavelength is higher than the first wavelength.

17. The device according to claim 16, wherein the filtering means comprise a high-pass filter having a limit wavelength located between the first wavelength and the main emission wavelength, and a band-stop filter corresponding to the second wavelength.

18. The device according to claim 13, wherein the second wavelength is lower than the first wavelength.

19. The device according to claim 18, wherein the filtering means comprise a high-pass filter having a limit wavelength located between the first wavelength and the main emission wavelength.

20. A method for displaying an object using a fluorescence reflection imaging device according to claim 13, comprising a step of marking cells by the marking fluorophore and alternately a first step of activation of the first light source and acquisition of a fluorescence image, and a second step of activation of the second light source and acquisition of a background noise image.

21. The method according to claim 20, comprising adjustment of a fluorescence image and of a background noise image so that an image area corresponding to a predetermined area not marked by the marking fluorophore presents the same luminosity on the fluorescence image and on the background noise image.

22. The method according to claim 21, wherein adjustment of a fluorescence image and of a background noise image comprises adjustment of the durations of the first and second steps.

23. The method according to claim 20, comprising subtraction of the fluorescence image and of the background noise image.

24. The method according to claim 20, comprising division of the fluorescence image by the background noise image.
